(19) United States
(12) Patent Application Publication
(10) Pub. No.: US 2023/0284928 A1
(43) Pub. Date: Sep. 14, 2023

(54) DETERMINING A LOCATION OF AN APPARATUS IN AN MRT SYSTEM

(71) Applicant: Siemens Healthcare GmbH, Erlangen (DE)

(72) Inventor: Stefan Popescu, Erlangen (DE)

(21) Appl. No.: 18/105,381

(22) Filed: Feb. 3, 2023

(30) Foreign Application Priority Data
Feb. 3, 2022 (EP) 22154982.7

(51) Int. Cl. A61B 5/055; A61B 5/00

(52) U.S. Cl. CPC A61B 5/055; A61B 5/68

(57) ABSTRACT

A method for determining a location of an apparatus inside an imaging volume of an MRT system surrounded by a basic field magnet for creating a static basic magnetic field along a longitudinal axis and by a gradient coil is provided. The apparatus has a first conductor loop that runs within a loop plane. The method includes creating a magnetic alternating field in the imaging volume using the gradient coil. At least one measured value that depends on an induction voltage that is induced by a component of the alternating field at right angles to the longitudinal axis in the at least one conductor loop is determined using the at least one first conductor loop. A location of the apparatus inside an imaging volume is determined at least partly as a function of the at least one measured value and a predetermined magnetic field model for the gradient coil.

This application claims the benefit of European Patent Application No. EP 22154982.7, filed Feb. 3, 2022, which is hereby incorporated by reference in its entirety.

### BACKGROUND

The present embodiments relate to a method for determining the location of an apparatus inside an imaging volume of a magnetic resonance tomography (MRT) system, where the imaging volume is surrounded by a field magnet for creation of a static basic magnetic field along a longitudinal axis and by a gradient coil of the MRT system. The present embodiments are further directed to a corresponding MRT system.

Magnetic resonance tomography (MRT) systems are imaging apparatuses that use a strong external magnetic field in order to align nuclear spins of an object to be examined and to excite the nuclear spins to precess around a corresponding alignment by application of a radio frequency (RF) excitation pulse. The precession or the transition of the spins from the excited state into a state with lower energy creates an electromagnetic alternating field as a response, which may be detected as an MR signal via a receive antenna.

With the aid of magnetic gradient fields, a position encoding may be impressed onto the signals, which subsequently allows the signal received to be assigned to a volume element of the examination object. The received signal may then be evaluated in order, for example, to create a pictorial representation of the examination object.

In many MRT applications, it is advantageous to know the location (e.g., the position and/or orientation) of an apparatus within the imaging volume defined by the gradient (e.g., with regard to the anatomy of the patient). The location of the patient in the imaging volume may be defined or determined, for example, by visual markers or the like, so that it is desirable to determine the location of the apparatus as accurately as possible. The apparatus may, for example, involve local MR receive coils that are arranged directly on the patient, such as head coils, knee coils, and so forth. The apparatus may, however, also involve a device for medical treatment of the patient, such as a catheter, a surgical instrument, a biopsy needle, a robot arm, and so forth.

The movement of the patient during an MRT examination is a known problem (e.g., in MRT examinations that take a longer time). The movement of the patient may change the measured signals and cause image artifacts, which may prevent or obscure the recognition of significant features (e.g., of the radiological findings). It is against this background, for example, that the determination of the location of the apparatus is advantageous.

One method for recognizing the position of a receive coil uses a Hall sensor integrated into the coil electronics that measures the local intensity of the static magnetic field. Outside the imaging volume, the static magnetic field is very inhomogeneous and has strong static field gradients. The measured values of the Hall sensor may be used to establish the position of the receive coil when the receive coil and the patient are moved from the patient table into the imaging volume. As soon as the receive coil is located within the imaging volume characterized by a very homogeneous magnetic field, however, the signal of the Hall sensor remains essentially constant, even when the coil position changes within the imaging volume. The result of this is that the change in the location of the receive coil because of patient movements may remain undetected.

In other methods, a camera is used in order to recognize the position of the receive coil on the body of the patient before the patient is brought with the patient table into the imaging volume. A further camera in the imaging volume may recognize the movement of the receive coil during the examination. However, this requires a clear line of sight between the camera and the receive coil, but this may be hindered, for example, by covers, other accessories, support elements, or dielectric pads for improving the RF environment or by the dimensions of the patient’s limbs.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a location of an apparatus within an imaging volume of a magnetic resonance tomography (MRT) system is reliably determined.

The present embodiments are based on the knowledge that a gradient coil creates a magnetic field in the inside of the gradient coil and thus in the inside of the imaging volume that, as well as components in parallel to the basic magnetic field, also has significant components at right angles thereto. These components are detected in accordance with the present embodiments by at least one conductor loop and are used to determine the location of the conductor loop and thereby of the apparatus.

In accordance with one aspect of the present embodiments, a method is specified for determining the location of an apparatus inside an imaging volume of an MRT system. The MRT system may have a field magnet for creating a static basic magnetic field along a longitudinal axis of the MRT system as well as a gradient coil that surrounds the imaging volume. The apparatus has at least one first conductor loop that runs within a first loop plane. Using the gradient coil, a magnetic alternating field is created in the imaging volume, and a first measured value is determined using the at least one first conductor loop, which depends on a first induction voltage that is induced in the at least one first conductor loop by a first component of the alternating field at right angles to the longitudinal axis. A location of the apparatus inside the imaging volume is at least partly determined as a function of the at least one first measured value and a predetermined magnetic field model for the gradient coil.

The imaging volume, for example, involves a region within a magnetic resonance (MR) scanner of the MRT system (e.g., within the patient tunnel, which is also referred to as the bore), which is essentially defined by the gradient coil and, where necessary, by an RF transmit coil arranged radially within the gradient coil for sending radio frequency alternating fields. The imaging volume is thus, for example, defined as a volume region within which an object may in principle be imaged (e.g., when the RF send coil is also used as a receive coil). If a local MR receive coil is used, this is located, for example, within the imaging volume. This provides that the local MR receive coil does not define the imaging volume, but, where necessary, a further imaging volume inside the local MR receive coil.

The at least one conductor loop may, for example, be part of the local MR receive coil. In other words, the apparatus may then correspond to the local MR receive coil. In other forms of embodiment, however, the apparatus is configured as a dedicated sensor apparatus (e.g., independent of a possible local MR receive coil). The determination of the location of the apparatus in this case enables the location of further objects (e.g., of the patient or of a part of the patient’s body or of a medical tool) to be deduced, when a relative location of the further objects with regard to the apparatus is correspondingly predetermined or defined.

In order to determine the location of the apparatus at least partly, depending on the at least one measured value and the magnetic field model, a location of the at least one conductor loop is, for example, at least partly determined. The location of the at least one conductor loop may be the same as the location of the apparatus, or the location of the apparatus may be derived from the location of the at least one conductor loop.

The at least one conductor loop may also have a finite extent in a direction at right angles to the first loop plane. The fact that the at least one conductor loop runs within the first loop plane may be interpreted, such that all conductor loops of the at least one first conductor loop are parallel to one another and parallel to the loop plane in each case.

The location of the at least one conductor loop and accordingly the location of the apparatus may be determined in a predetermined coordinate system (e.g., a fixed-point coordinate system) with regard to the gradient coil. The longitudinal axis of the MRT system may, for example, be interpreted as the z direction of this coordinate system and corresponding directions at right angles to the z direction as the x and y axis of the coordinate system. Other reference systems may also be chosen, however, if these are advantageous for further use. The term location may be the combination of the three-dimensional position and three-dimensional orientation in the corresponding reference system. In other words, the location may also be referred to as the pose. For example, the location may be given by three-dimensional coordinates of a reference point of the apparatus and three orientation angles of a specific reference direction of the apparatus. In order to determine the location completely, the three-dimensional position and the three-dimensional orientation of the apparatus, for example, would thus have to be determined.

The fact that the location of the apparatus is determined at least partly in the method of the present embodiments may be understood such that the location is determined either completely or incompletely. An incomplete determination of the location may be understood as, for example, only individual coordinates of the three-dimensional position or individual angles of the three-dimensional orientation being determined, but not all three coordinates of the position and all three angles of the orientation. As an alternative, the part determination of the location of the apparatus may consist of one or more restrictions of the six degrees of freedom (e.g., of the three coordinates and of the three orientation means) being determined explicitly or implicitly, where the restriction goes beyond the arrangement of the at least one first conductor loop within the imaging volume.

For example, the at least part determination of the location thus enables it to be determined at what distance the reference point of the apparatus is located from the center of the imaging volume (e.g., to the longitudinal axis), in which angular range the corresponding orientation means are located, and so forth. Whether the location is determined completely or only partly or how much information is determined with regard to the location of the apparatus depends on whether and what additional information is available, as well as the at least one measured value and the magnetic field model for location determination. Even without such additional information, exclusively based on the at least one measured value and the magnetic field model, a part determination of the location is possible. A complete determination of the location as a rule however requires further information, which may be given, for example, as a result of constructional restrictions or determined by further sensor systems, such as possibly Hall sensors, further conductor loops, cameras, and so forth.

The magnetic field model may, for example, include the magnetic field created by the gradient coil inside the imaging volume in spatially resolved form and in all three spatial directions or in any event in three spatial dimensions (e.g., also in spatially and temporally resolved form). The magnetic field model thus, for example, depends on the geometrical and electrical characteristics of the gradient coil as well as possibly on the activation of the gradient coil. The magnetic field model may be determined, for example, by measurement of the magnetic field inside the imaging volume and/or by simulations and/or other computations and be stored on the MRT system (e.g., on at least one evaluation unit of the MRT system).

The gradient coil of an MRT system is configured so that, inside the imaging volume, primarily a magnetic field gradient of a magnetic field in the direction of the longitudinal axis may be created. The magnetic field gradient corresponds to a change in the magnetic field strength in one of the three spatial directions. An MRT system in this case has three such gradient coils, of which the magnetic field strength changes in each case in a different spatial direction. A magnetic field that is oriented exclusively along the longitudinal axis is only possible theoretically, however, for example, for infinitely large coils. In each actual implementation of a gradient coil, the magnetic field created by the coil inside the imaging volume always has components in all three spatial directions. Outstanding symmetry points in the exact center of the imaging volume may be an exception that, however, are likewise only of theoretical significance. This knowledge and the use of this knowledge about determining the location of the apparatus in the imaging volume of an MRT system are the basis of the present embodiments.

For example, it is possible, as a result of the circumstances outlined, independently of the orientation of the loop plane with regard to the longitudinal axis, to measure a corresponding induction voltage or a corresponding induction current and, with simultaneous knowledge of the magnetic field model, to make deductions about the location of the at least one conductor loop. In this way, the complexity of the determining the location of the apparatus (e.g., the complexity of the apparatus itself) may be reduced.

For example, the present embodiments make it possible for a local MR receive coil itself to be used for determining the location of the MR receive coil, although the conductor loops of a local MR receive coil are in parallel or not at right angles to the basic magnetic field. This makes possible a synergetic combination of the underlying functionality of the local MR receive coil (e.g., the detection of MR signals from the object to be examined), with the additional functionality of determining the location. In this case, the receive coil cannot only acquire radio frequency electromagnetic alternating fields of the send coil or the nuclear spin resonance signals in reaction to these, but also, the signals of the gradient coil that may possibly have a far lower frequency, which are to be created during the MRT examination to create the magnetic field gradients. It is thus not necessary for the magnetic alternating field of the gradient coil to be created dedicated to location determination. Although this is indeed possible, the gradient pulses created may, however, be used.

If the apparatus is configured as a dedicated sensor system with a location sensor and independently of a local MR receive coil, then the location sensor may be configured more simply by the present embodiments (e.g., by the location sensor only having the at least one first conductor loop), but not additionally further conductor loops at right angles to the loop plane. This does not, however, exclude a number of such location sensors being provided at different positions within the imaging volume for the most complete possible location determination of the apparatus.

If the location of the apparatus is known or at least narrowed down, then, for example, a user of the MRT system may automatically be informed as to whether the location deviates from a desired or optimal location. An algorithm that automatically selects one or more scan parameters, such as, for example, an acceleration factor R, a phase encoding direction, and so forth, as a function of the at least partly determined location of the apparatus may be employed, so that the given location or, for example, of the apparatus with regard to the patient, may be used in the optimal way. As an alternative or in addition, image reconstruction algorithms may also use the at least partly determined location in order, for example, through more accurate estimation of the coil sensitivity of the local MR receive coil, to improve the resulting image quality.

In accordance with at least one embodiment of the method, an MR image may be created as a function of an MR signal from an object to be examined in the imaging volume.

During the creation of the MR image, the at least partly determined location of the apparatus may be taken into consideration automatically or manually. For example, an MR recording may be repeated or partly repeated when the location does not correspond to a predetermined requirement or to a predetermined expectation. Movement compensation algorithms may be executed automatically depending on the at least partly determined location in order to create the MR image.

To create the MR signal, a known MR sequence may be applied. For example, a global RF send coil that surrounds the imaging volume radiates corresponding RF pulses into the imaging volume, and using the gradient coil, a sequence of magnetic field gradients is created in the imaging volume. Through this, a nuclear spin resonance is brought about in the object to be examined, and radio frequency MR signals produced may then be detected by the RF send coil, if this is also used as a receive coil, and/or by one or more further local MR receive coils.

In accordance with at least one embodiment, the MR signal from the object to be examined is detected in the imaging volume using the at least one first conductor loop, and the MR image is created as a function of the MR signal.

In other words, the at least one first conductor loop is used not only for creation of the at least one measured value and thus for at least partly determining the location of the apparatus, but also as a regular local MR receive coil. The apparatus or the at least one conductor loop is, for example, part of a local MR receive coil.

In principle, the MR signal is also detected via a corresponding induction voltage in the at least one conductor loop. The induction voltages resulting from the MR signal and because of the magnetic alternating field of the gradient coil, which are employed for determination of the at least one measured value, may be separated from each other in time or in another way. For example, use may be made of the fact that the frequency of the signals created by the send coil and, accordingly, the frequency of the MR signals are greater by a multiple than a frequency of the magnetic alternating fields created by the gradient coil. Thus, for example, a frequency filtering may be carried out in order to separate from one another the detection of the MR signal from the detection of the magnetic alternating field created by the gradient coil.

The frequency of the MR signal in this case, for example, corresponds to the corresponding Larmor frequency of the atomic nuclei used for imaging. This lies, for example, in a frequency range of 1 MHz to 500 MHz, depending on the basic magnetic field strength of the field magnet of the MRT system. The pulsed gradient fields of the gradient coil (e.g., of the magnetic alternating field created by the gradient coil) has a frequency in the range of a few kHz or a few 10 kHz.

In accordance with at least one embodiment, in which the MR signal is detected by the at least one first conductor loop, for determination of the at least one first measured value, the MR signal or the further induction voltage or a corresponding signal resulting from the MR signal is suppressed (e.g., by a filter circuit).

What is thus achieved by this is that the at least one measured value merely reflects the magnetic alternating field of the gradient coil, but not the MR signal.

For determination or detection of the MR signal, the induction voltage resulting from the magnetic alternating field of the gradient coil may be suppressed, for example, by the filter circuit or a further filter circuit.

In this way, it may thus be provided that the detection of the MR signal is not influenced by the magnetic alternating field of the gradient coil.

For example, the at least one first measured value and the MR signal may be detected by different measurement channels or receive channels, where the filter circuit and/or the further filter circuit is implemented in the corresponding measurement channels.

In accordance with at least one embodiment, the apparatus is positioned in the imaging volume such that the loop plane is parallel to the longitudinal axis (e.g., is at least approximately parallel to the longitudinal axis).

In this case, the loop plane may, for example, be considered as at least approximately parallel to the longitudinal axis when the angle between a normal direction, which is at right angles to the loop plane, and the longitudinal axis, is at least approximately equal to 90° (e.g., greater than 60 degrees and less than 120 degrees, greater than 70° and less than 110°, or greater than 80° and less than 100°).

This may be the case, for example, when the apparatus of a local MR receive coil corresponds to or is part of such a plane.

In accordance with at least one embodiment, the apparatus has at least one second conductor loop that runs within a second loop plane that is different from the first loop plane. Using the at least one second conductor loop, at least one second measured value is determined that depends on a second induction voltage that is induced by a second component of the alternating field at right angles to the longitudinal axis in the at least one second conductor loop. The location of the apparatus is determined at least partly as a function of the at least one measured value, the at least one second measured value, and the magnetic field model for the gradient coil.

For example, in such forms of embodiment, the at least one first conductor loop and the at least one second conductor loop may be part of a local MR receive coil.

For example, the apparatus is positioned in the imaging volume such that the second loop plane is at least approximately parallel to the longitudinal axis and the first loop plane, for example, is likewise at least approximately parallel to the longitudinal axis.

For example, the location of the at least one first conductor loop relative to the at least one second conductor loop may be known or be predetermined as a fixed location. Thus, for at least part determination of the location of the apparatus, the location of the at least one first conductor loop and the location of the at least one second conductor loop may be determined as described. Thus, the predetermined or known relative location in relation to one another enables the location of the apparatus to be determined more precisely thereby or narrowed down more fully.

In corresponding developments, a plurality of further conductor loops may be used in a similar way to the at least one first conductor loop and the at least one second conductor loop in order to make possible a determination of the location of the apparatus that is as accurate or complete as possible, where the further conductor loops may, for example, be part of the local MR receive coil.

In accordance with at least one embodiment, the apparatus has at least one third conductor loop that runs within a third loop plane. Using the at least one third conductor loop, at least one third measured value is determined. The at least one third measured value depends on a third induction voltage that is induced by a third component of the alternating field at right angles to the longitudinal axis in the at least one third conductor loop. A first location of the at least one first conductor loop inside the imaging volume is determined at least in part as a function of the at least one first measured value and the magnetic field model, and a third location of the at least one third conductor loop inside the imaging volume is determined at least in part as a function of the at least one third measured value and the magnetic field model. A relative location of the at least one third conductor loop with regard to the at least one first conductor loop is determined as a function of the first location and as a function of the third location.

Thus, in such forms of embodiment, the relative location of the third conductor loop and the first conductor loop, for example, is not known or not precisely known in advance but may be determined or approximately determined in the way described. Such forms of embodiment are likewise, for example, advantageous when the local MR receive coil contains the at least one first conductor loop and the at least one third conductor loop and is configured, for example, as a flexible surface coil. Such flexible surface coils where necessary adapt themselves to the surface of the patient or the like, so that the individual conductor loops do not have an orientation known per se relative to each other. In this way, the location or the form of the surface of the flexible surface coil may be determined.

In accordance with a further aspect of the present embodiments, an MRT system is also specified that has a field magnet for creating a static basic magnetic field along a longitudinal axis and a gradient coil. The field magnet and the gradient coil surround an imaging volume of the MRT system. The MRT system has an apparatus with at least one first conductor loop, where the at least one first conductor loop runs within a first loop plane. The MRT system has a control unit that is configured to activate the gradient coil, to create a magnetic alternating field in the imaging volume. The MRT system has a measurement unit that is connected to the at least one first conductor loop. The measurement unit is configured, depending on a first induction voltage that is induced by a component of the alternating field at right angles to the longitudinal axis in the at least one first conductor loop, to determine at least one first measured value. The MRT system has at least one evaluation unit that is configured to determine, at least in part, a location of the apparatus inside the imaging volume as a function of the at least one first measured value and a predetermined magnetic field model for the gradient coil.

In different forms of embodiment, the control unit, the measurement unit, and/or the at least one evaluation unit may be provided separately from one another or also be combined partly or completely.

In accordance with at least one embodiment, the MRT system has a local MR receive coil arrangement that contains the apparatus.

In accordance with at least one embodiment, the local receive coil arrangement is configured as a flexible surface coil array.

The at least one first conductor loop then corresponds to a surface coil of the surface coil array.

In accordance with at least one embodiment, the MRT system has a device for medical treatment of a patient. The at least one first conductor loop and the device have a predetermined spatial location in relation to one another.

The device may, for example, involve a biopsy needle, a catheter, a surgical instrument, a robot arm, and so forth.

In accordance with at least one embodiment, the apparatus has a tuning capacitance (e.g., a tuning capacitor) that is arranged respectively between a first terminal of the at least one first conductor loop and a second terminal of the at least one first conductor loop. The apparatus has an inductive component that is arranged electrically in parallel to the tuning capacitance.

Such a form of embodiment of the apparatus is, for example, advantageous when the apparatus is part of the local MR receive coil arrangement or is the same as the local MR receive coil arrangement.

The induction voltage is present, for example, between the first terminal and the second terminal of the at least one conductor loop.

The tuning capacitance may be realized as a tuning capacitor (e.g., as a corresponding electronic component), or as a parasitic capacitance between conductor segments of the at least one first conductor loop.

For example, the apparatus may have a number of tuning capacitances that are arranged between the first terminal and the second terminal. In this case, the apparatus (e.g., for each tuning capacitance) has a corresponding assigned inductive component that is connected electrically in parallel to the tuning capacitance.

Without the inductive component, the at least one conductor loop, because of the tuning capacitance, would be non-conducting for direct current, for example, or would have a very high impedance with regard to low-frequency alternating currents. Through the inductive component, the conductivity at low frequencies is increased. With respect to the radio frequency MR signals, the inductance of the inductive component effectively acts as a resistance, so that, as a result of the parallel connection to the tuning capacitance, there is no significant influence during the detection of the MR signal.

In different forms of embodiment, the apparatus may also have a detuning capacitance (e.g., a detuning capacitor) that is arranged respectively between the first terminal and the second terminal. The apparatus has a further inductive component that is connected electrically in parallel with the tuning capacitance. Thus, the detuning capacitance also has no or hardly any effect at low frequencies, whereas the further inductive component does not have an effect or has no significant effect at high frequencies.

In accordance with at least one form of embodiment, the measurement unit has an amplifier that is connected to the first terminal and the second terminal (e.g., directly or indirectly) and is configured, at an output of the amplifier that is connected to the at least one evaluation unit, to provide the at least one measured value.

For example, the amplifier has a first input and a second input. The first input is connected to the first terminal, and the second input to the second terminal.

In accordance with at least one form of embodiment, the measurement unit or the apparatus has a further amplifier that is likewise connected to the first terminal and the second terminal and is configured, at an output of the further amplifier that is likewise connected to the at least one evaluation unit, to provide the MR signal or a measurement signal dependent on the MR signal. The amplifier and the further amplifier may thus, for example, be corresponding parts of a first and a second measurement channel that detect different currents acquired with the at least one conductor loop.

In accordance with at least one form of embodiment, the measurement unit has a filter circuit that is arranged between the first terminal and a first input of the amplifier, and also between the second terminal and a second input of the amplifier. The filter circuit is configured to suppress an MR signal acquired by the at least one conductor loop.

The filter circuit, for example, has a first input that is connected to the first terminal, and a first output that is connected to the first input of the amplifier. Further, the filter circuit, for example, has a second input that is connected to the second terminal, and a second output that is connected to the second input of the amplifier.

The filter circuit may, for example, be configured as a lowpass filter or bandpass filter. In any event, the filter circuit is tuned to the gradient coil, the basic magnetic field, or the activation of the gradient coil, such that the filter circuit essentially lets frequencies that correspond to the alternating magnetic alternating field of the gradient coil pass, whereas the filter circuit essentially suppresses frequencies that correspond to the MR signal.

For example, the apparatus may have a further filter circuit that is arranged between the first terminal and a first input of the further amplifier and also between the second terminal and a second input of the further amplifier. The further filter circuit is configured to suppress the signal created by the magnetic alternating field of the gradient coil in the at least one first conductor loop.

The second filter circuit may be configured, for example, as a highpass filter or as a further bandpass filter. The further filter circuit is thus, for example, structured complementarily to the filter circuit. For example, the first terminal of the at least one conductor loop may be connected to a first input of the further filter circuit, and the second terminal of the at least one conductor loop may be connected to a second input of the further filter circuit. A first output of the further filter circuit is, for example, connected to a first input of the further amplifier, and a second output of the further filter circuit is connected to a second input of the further amplifier.

Further forms of embodiment of the MRT system follow on directly from the different embodiments of the method, and vice versa. For example, individual features and corresponding explanations with regard to the different forms of embodiment for the method may be transferred by analogy to corresponding forms of embodiment of the MRT system, and vice versa. For example, the MRT system of the present embodiments is embodied or programmed for carrying out a method of the present embodiments. For example, the MRT system carries out the method of the present embodiments.

In accordance with a further aspect of the present embodiments, an apparatus for location determination for an MRT system is also specified. The apparatus has at least one first conductor loop that runs within a first loop plane. The apparatus also has a measurement unit that is connected to the at least one conductor loop and is configured, depending on a first induction voltage that is induced in the at least one first conductor loop, to determine at least one first measured value.

In accordance with at least one form of embodiment of the apparatus, this has the tuning capacitance and the inductive component, as described above.

Further forms of embodiment of the apparatus of the present embodiments follow on from the different forms of embodiment of the MRT system of the present embodiments, as well as from the method of the present embodiments.

A computing unit may, for example, be understood as a data processing device that contains a processing circuit. The computing unit may thus, for example, process data for carrying out the computing operations. This may also include operations for carrying out indexed accesses to a data structure (e.g., a Look-Up Table (LUT)).

The computing unit may, for example, contain one or more computers, one or more microcontrollers, and/or one or more integrated circuits (e.g., one or more application-specific integrated circuits (ASICs), one or more Field-Programmable Gate Arrays (FPGAs), and/or one or more systems on a chip (SoCs)). The computing unit may also contain one or more processors (e.g., one or more microprocessors, one or more Central Processing Units (CPUs), one or more graphics processing units (GPUs), and/or one or more signal processors, such as one or more Digital Signal Processors (DSPs)). The computing unit may also include a physical or a virtual network of computers or other computing units.

In different forms of embodiment, the computing unit includes one or more hardware and/or software interfaces and/or one or more memory units.

A memory unit may be configured as a volatile data memory (e.g., as Dynamic Random Access Memory (DRAM) or Static Random Access Memory (SRAM), or as nonvolatile data memory, such as Read-Only Memory (ROM), as Programmable Read-Only Memory (PROM), as Erasable Read-Only Memory (EPROM), as Electrically Erasable Read-Only Memory (EEPROM), as flash memory or flash EEPROM, as Ferroelectric Random Access Memory (FRAM), as Magnetoresistive Random Access Memory (MRAM), or as Phase-Change Random Access Memory (PCRAM)).

The at least one evaluation unit, the control unit, and/or the measurement unit of the MRT system of the present embodiments may include one or more computing units in accordance with this understanding, or one or more computing units of the MRT system may include the at least one evaluation unit, the control unit, and/or the measurement unit.

Within the framework of the present disclosure, when reference is made to a component of the MRT system (e.g., the control unit, the measurement unit, or at least one evaluation unit of the MRT system) being configured, designed, or the like to carry out or realize a specific function, achieve a particular effect, or serve a particular purpose, this may be understood such that the component, above and beyond the principle or theoretical usability or suitability of the component for this function, effect, or this purpose, through a corresponding adaptation, programming, physical embodiment, and so forth, is in a position in concrete terms and actually to carry out or to realize the function, to achieve the effect, or to serve the purpose.

A connection between two electrical or electronic components may, unless explicitly stated otherwise, be understood such that an electrical connection exists between the components or may be established by actuation of one or more switching elements. For example, the components may be connected to one another directly or indirectly, unless stated otherwise. In such cases, a direct connection may be understood as, apart from the optional one or more switching elements, no further electrical or electronic components being arranged between the components, while an indirect connection may be understood as, in addition to the optional one or more switching elements, one or more further electrical or electronic components, such as resistors, capacitors, coils, and so forth being arranged between the components.

Further features of the present embodiments emerge from the claims, the figures, and the description of the figures. The features and combinations of features given above in the description, as well as the features and combinations of features given below in the description of the figures and/or in the figures, may be included not only in the specified combination in each case, but also in other combinations of the invention. For example, versions and combinations of features may not have all features of an originally formulated claim. Above and beyond this, versions and combinations of features may go beyond the combinations of features set out in the references of the claims or that deviate therefrom.

### BRIEF DESCRIPTION OF THE DRAWINGS

The invention will be explained in greater detail below with the aid of concrete exemplary embodiments and associated schematic diagrams. In the figures the same elements or elements with the same function are provided with the same reference character. The description of the same elements or elements with the same function may not be repeated with regard to different figures. In the figures:

FIG. 1 shows a schematic diagram of an embodiment of a magnetic resonance tomography (MRT) system;

FIG. 2 shows a schematic diagram of a magnetic field;

FIG. 3 shows a schematic diagram of an apparatus of a further embodiment of an MRT system;

FIG. 4 shows a schematic diagram of an apparatus of a further embodiment of an MRT system;

FIG. 5 shows a schematic diagram of an apparatus of a further embodiment of an MRT system;

FIG. 6 shows a schematic diagram of an apparatus of a further embodiment of an MRT system;

FIG. 7 shows a schematic diagram of an apparatus of a further embodiment of an MRT system;

FIG. 8 shows a schematic diagram of an apparatus of a further embodiment of an MRT system;

FIG. 9 shows a schematic diagram of an apparatus of a further embodiment of an MRT system; and

FIG. 10 shows a schematic diagram of possible basic forms of a local MR receive coil.

### DETAILED DESCRIPTION

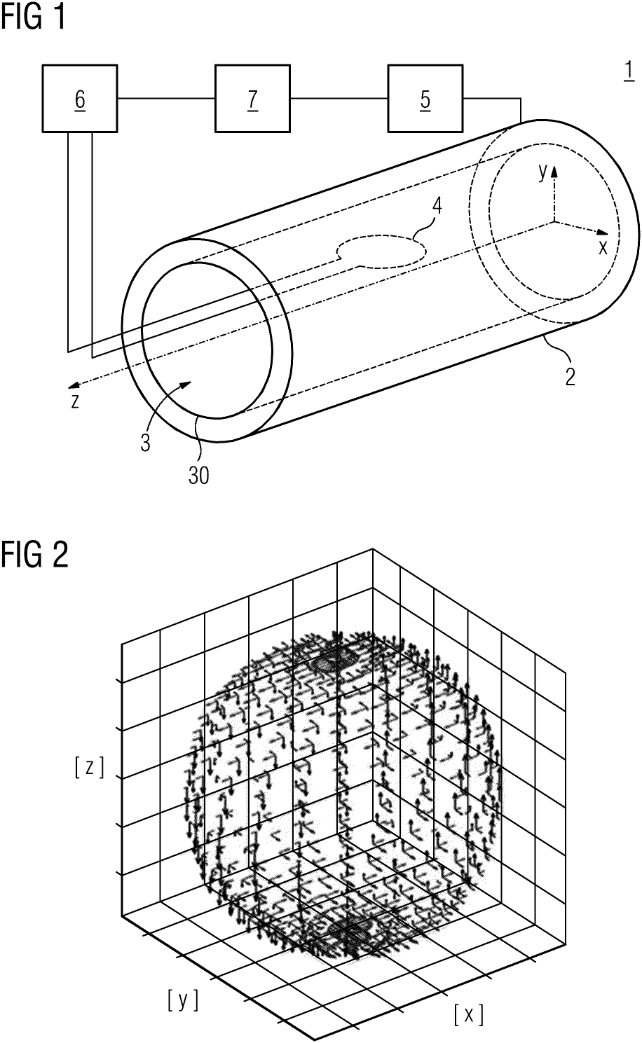

FIG. 1 is a schematic of an embodiment of a magnetic resonance tomography (MRT) system 1.

The MRT system 1 also has a field magnet (not shown) that creates a static magnetic field for alignment of nuclear spins of a sample (e.g., of a patient) in an imaging volume 3 in the z direction that may be referred to as the longitudinal axis of the MRT system. The imaging volume 3 is characterized by a very homogeneous static magnetic field in the z direction. The field magnet may, for example, involve a superconducting magnet that may provide magnetic fields with a magnetic flux density of up to 3 T or more. For smaller field strengths, however, permanent magnets or electromagnets with normally conducting coils may also be used.

Further, the MRT system 1 has a gradient coil 2 and also a control unit 5 for activating the gradient coil 2 that is configured, for spatial differentiation of the imaging regions acquired in the imaging volume 3, to overlay the static magnetic field with magnetic fields of which the amount may change, depending on location, along all three spatial directions x, y, z. The gradient coil 2 may, for example, be configured as a coil of normally conducting wires.

The MRT system 1 may, for example, have a body coil 30 as a send antenna that is configured to radiate a radio frequency signal supplied via a signal line into the imaging volume 3.

The control unit 5 may supply the gradient coil 2 and the body coil 30 with different signals. The control unit 5 may, for example, have a gradient controller that is configured to supply the gradient coil 2 via supply lines with variable currents that, temporally coordinated, may provide the desired gradient fields in the imaging volume 3.

The control unit 5 may also have a radio frequency unit that is configured to create radio frequency pulses or excitation pulses with predetermined temporal waveforms, amplitudes, and spectral power distribution for exciting a magnetic resonance of the nuclear spin in the patient. In such cases, pulse powers in the range of kilowatts may be employed. The excitation pulses may be radiated into the patient via the body coil 30 or via one or more local send antenna. The control unit 5 may also contain a controller that may communicate via a signal bus with the gradient controller and the radio frequency unit.

The body coil 30, in some forms of embodiment, may also be used to receive resonance signals (e.g., magnetic resonance (MR) signals) emitted by the patient, and output the resonance signals via a signal line. The body coil 30 in such forms of embodiment may thus serve as both a receive antenna and also as a send antenna.

Optionally, a local MR receive coil (not shown), also referred to as a local coil, of the MRT system 1 may be arranged in the immediate vicinity of the patient, which may be linked via a connecting line to a measurement unit 6. The measurement unit 6 may also be part of the control unit 5. Depending on form of embodiment, the local coil, as an alternative or in addition to the body coil 30, may serve as a receive antenna.

The MRT system 1 may also have an evaluation unit 7 that is connected to the control unit 5 (e.g., to the radio frequency unit). The evaluation unit 7 may evaluate the MR signals and, based thereon, reconstruct an MR image according to known methods. The control unit 5 may also be part of the evaluation unit 7.

The MRT system 1 has an apparatus with at least one conductor loop 4 that runs within a loop plane and, for example, is arranged in the imaging volume 3 such that the loop plane is essentially oriented in parallel to the z direction.

As described, the gradient coil 2, activated by the control unit 5, creates a magnetic alternating field in the imaging volume 3. This magnetic alternating field generally has magnetic field components in all three spatial directions x, y, z. As a consequence, an induction voltage is brought about in the at least one conductor loop 4, even when the loop plane is oriented essentially in parallel to the z direction.

The measurement unit 6 is connected to the at least one conductor loop 4 and is configured, depending on the induction voltage, to determine at least one measured value. The evaluation unit 7 is configured to determine, at least partly, a location of the apparatus inside an imaging volume 3 as a function of the at least one measured value and a predetermined magnetic field model for the gradient coil 2.

In forms of embodiment with a local MR receive coil, this may include the apparatus or the at least one conductor loop 4. The location of the apparatus then thus corresponds to the location of the local MR receive coil. As an alternative, the apparatus may be used as a self-contained location sensor, with which, for example, the location of a medical device (not shown) in the imaging volume 3 may be at least partly determined, especially when the relative location of the medical device in relation to the at least one conductor loop 4 is known.

An example for the magnetic field model is shown schematically in FIG. 2. Contrary to the usual simplified assumption, the gradient fields created by the gradient coil 2 in the imaging volume 3 are not aligned exactly parallel to the direction of the static magnetic field (e.g., the z direction). Instead, the gradient coil 2 creates additional field components that are aligned orthogonally to z (e.g., along the x or y axis), and the amplitude of which is also comparable with the z component. Since the components of the alternating field along the x or y axis are very much smaller than the static basic magnetic field, these components may be ignored for the regular MRT imaging. For this reason, the components of the alternating field along the x-axis and the y-axis have in the past also not been taken into consideration for other possible applications. Since the components of the alternating field, unlike the static basic magnetic field, are time-dependent, the components of the alternating field contribute significantly to the induction in the conductor loop 4 and may therefore be used for location determination in accordance with the present embodiments.

FIG. 2, by way of example, shows the cartesian components of the magnetic field, as are acquired inside the gradient coil 2, which is operated in the static mode (e.g., with a constant current). No basic magnetic field of the field magnet is present. At each sampling point, three field values that correspond to the three orthogonal field components Bx, By and Bz have been measured with a vector magnetometer that was attached to a robot arm and was positioned at 480 spatial positions that are distributed over the surface of a sphere. Based on these measured values, with the aid of a calibrated magnetic field model, the magnetic field at any given place within the imaging volume 3 may be computed. The at least one conductor loop 4, which is located in the imaging volume 3 of the MRT system 1, thus acquires a signal induced by the pulsed gradient fields, even if the loop plane is essentially oriented in parallel to the z direction.

Through the present embodiments, it is not absolutely necessary to use additional sensors for determination of the location of a local MR receive coil. Instead, the conductor loops of the local MR receive coil already present may be employed both for detection of the weak radio frequency MRT signals and also for detection of the signals induced by the gradient pulses in the low-frequency range.

The voltage that is induced in a conductor loop when the magnetic flux through the region surrounded by the conductor loop changes is produced by an integration of the change of the magnetic vector field B over the surface A enclosed by the loop (e.g., by application of Faraday’s law of induction):

$$\text{U} = \int \frac{\text{d} \vec{\text{B}}}{\text{dt}} ⋅ \text{d} \vec{\text{s}}$$

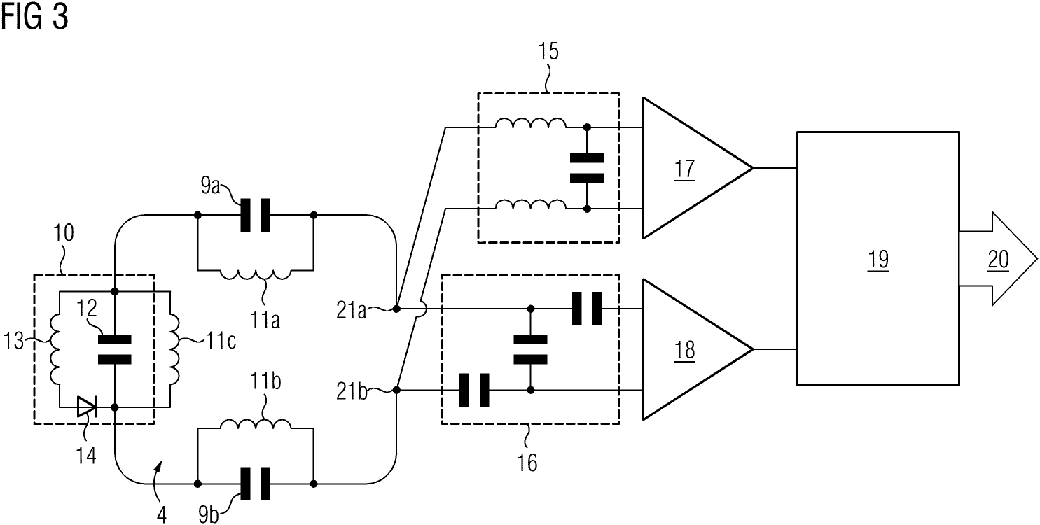

Shown in FIG. 3 to FIG. 5 are schematic implementations of the apparatus with the at least one conductor loop 4 for different embodiments of the MRT system 1 (e.g., of the MRT system 1 from FIG. 1).

Through these apparatuses, the MRT system 1 is capable of simultaneously receiving the radio frequency MR signals and the low-frequency signals for location determination induced by pulsing gradient fields.

A multi-channel MR receive coil may be embodied, for example, as a two-dimensional flexible array that consists of a number of receive elements, such as 2 to 32 or even 64 receive elements. Such a receive element is shown in FIG. 3 to FIG. 5. The receive element has the at least one conductor loop 4 (e.g., configured as at least one copper loop), as well as tuning devices (e.g., tuning capacitors 9a, 9b) that are arranged between a first terminal 21a and a second terminal 21b of the at least one conductor loop 4. Detuning devices 10 may also be provided, which, for example, contain a detuning capacitor 12 and a series circuit arranged in parallel thereto with a detuning inductance 13 and a diode 14.

A preamplifier circuit 18 that is connected on an input side via a matching circuit 16 to the terminals 21a, 21b and on an output side to an analog-to-digital converter 19, which may be linked via a data bus 20 to the evaluation unit 7 or a computer, may be provided. The tuning capacitors 9a, 9b are, for example, distributed along the at least one conductor loop 4 in order to reduce the electrical fields that otherwise occur over long line conductor segments and may possibly lead to dielectric losses and thus to a reduced signal-to-noise ratio. The capacitances of the tuning capacitors 9a, 9b are, for example, tuned such that the tuning capacitors 9a, 9b resonate with the inductance of the at least one conductor loop 4 at the Larmor resonant frequency of the MRT system 1, which, depending on field strength, may have a high frequency, for example, in the range of 1 MHz to 500 MHz.

In parallel to the tuning capacitors 9a, 9b and to the detuning capacitor 12 in each case is an inductive component 11a, 11b, 11c, so that the low-frequency signals induced by the gradient fields in the range of a few kHz may be acquired. The inductivity of inductive components 11a, 11b, 11c is chosen so that, for the induced radio frequency MR signals, these have a high impedance and in practice correspond to an open circuit. By contrast, the electrical impedance of the inductive components 11a, 11b, 11c at low frequencies is essentially equivalent to a short circuit, which closes the at least one conductor loop 4 for the signals induced by the pulsing gradient fields. The values of these inductances depending on the Larmor frequency may, for example, lie in the range of a few hundred µH to many mH.

In some forms of embodiment, a signal preamplifier 17 may be connected on the input side via a filter circuit 15 that may be configured, for example, as a lowpass filter, to the terminals 21a, 21b and, on the output side, to a further input of the analog-to-digital converter 19 or to a further analog-to-digital converter (not shown). The signals induced by the pulsed gradients and acquired by the at least one conductor loop 4 may then be read out via the data bus 20 and be further used by the signal processing algorithms, in order to extract the information about the location of the at least one conductor loop 4. In a similar way, the location of further receive elements may also be determined, and thus, the form of the flexible multi-channel MR receive coil may be described.

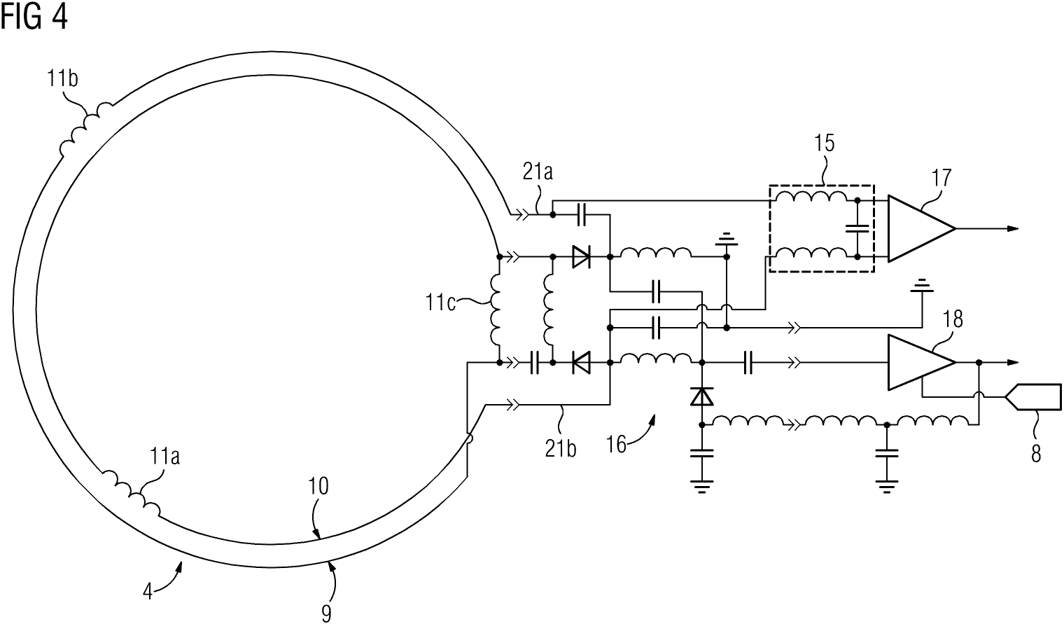

FIG. 4 shows schematically a receive element of an apparatus in a further form of embodiment of the MRT system 1 for a newer type of MR receive coil that uses distributed tuning capacitances 9 instead of discrete capacitors, which are formed by parasitic capacitances between conductor segments of at least one conductor loop 4. In this case, the separate conductor segments are effectively short circuited by the inductive components 11a, 11b, 11c for lower frequencies, so that the conductor segments form a double loop. Also indicated in FIG. 4 is a supply voltage 8 for the preamplifier circuit 18.

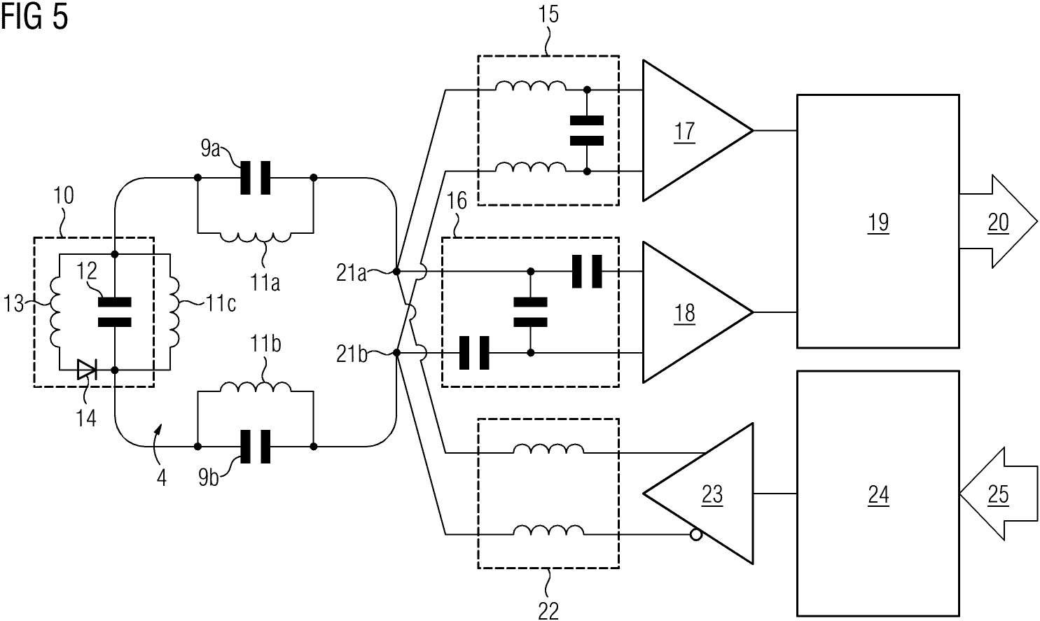

FIG. 5 shows schematically a receive element of an apparatus in a further embodiment of the MRT system 1. The receive element is based on the receive element shown in FIG. 3.

The receive element of FIG. 5, in corresponding forms of embodiment, fulfills a function referred to as local shimming. For example, the evaluation unit 7 or another computer connected to the receive element, for example, may adapt a direct current through the at least one conductor loop 4, in order to compensate for local inhomogeneities of the static magnetic field. The desired digital value of the direct current is, for example, transferred via a further data bus 25 to a further digital-to-analog converter 24, of which the output outputs a corresponding signal to a constant current driver 23. The constant current driver 23 may transmit the signal, for example, via a further lowpass filter 22 to the at least one conductor loop 4.

The further lowpass filter 22 in this case is, for example, configured so that the further lowpass filter 22 transmits the direct current value from the constant current driver 23 to the at least one conductor loop 4 and, in doing so, blocks the low-frequency alternating current signals induced by the pulsed gradient fields as well as the radio frequency MR signals. The filter circuit 15 may then, for example, be configured as a bandpass filter that may let the alternating current signals induced by the pulsing gradient fields pass and suppresses the direct current component and also the radio frequency MR signals. In a similar way, a receive element with distributed tuning capacitances 9 may be adapted as in FIG. 4.

The spatial location of objects within the imaging volume 3 may be determined, for example, through the processing of the signals, which are dependent on orthogonal coils that are attached to the object as a function of the voltages induced by the pulsing gradient fields. One method may begin with an initial estimation and then iteratively adapt the object position and alignment until the specific convergence criteria are fulfilled. In another method, a translation matrix is calibrated in a pre-training step, in which a test object moves in steps, an image volume is acquired for each step at the same time, and the gradient activity is measured. These methods may also be combined with the aid of the method of the present embodiments, of the MRT system 1 of the present embodiments, or of the apparatus of the MRT system 1 of the present embodiments in order to achieve the advantages explained.

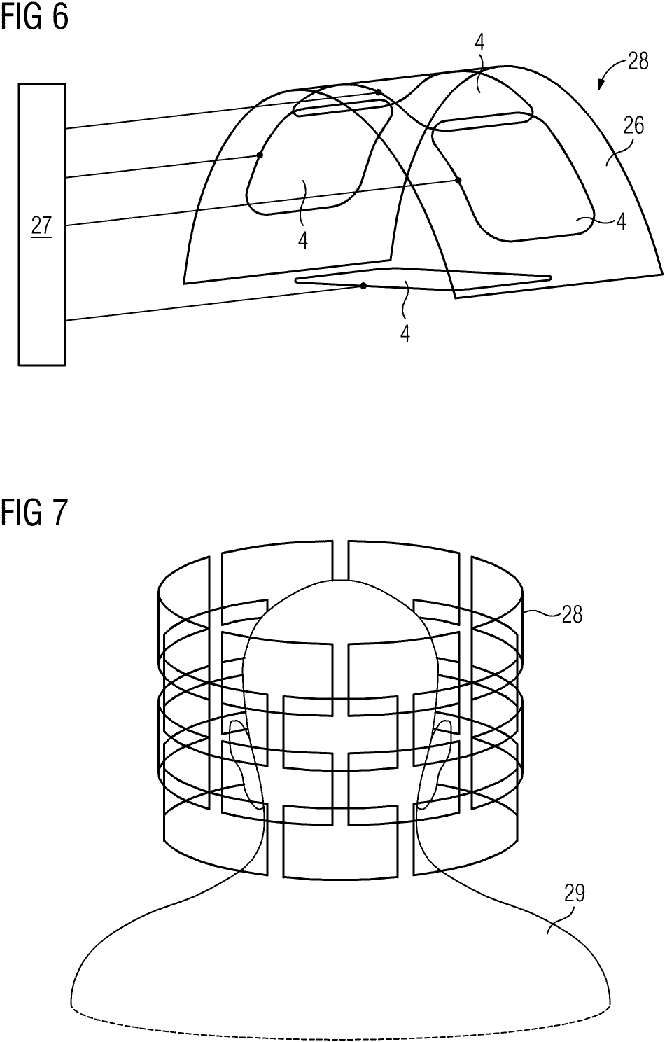

This is a method that may be used for recognizing the shape and position of local flexible MR receive coils 28, as shown schematically in FIG. 6 to FIG. 9 and for recognizing the patient movement. In this case, the advantages already explained may be utilized.

Flexible MR receive coils 28, for example, have a relatively large number of receive elements with corresponding conductor loops that, when attached to the body of the patient 29, may change their shape, as shown in FIG. 6 to FIG. 9, in order to follow the contours of the body of the patient 29, and that possibly also move because of the breathing movement or the heartbeat of the patient 29.

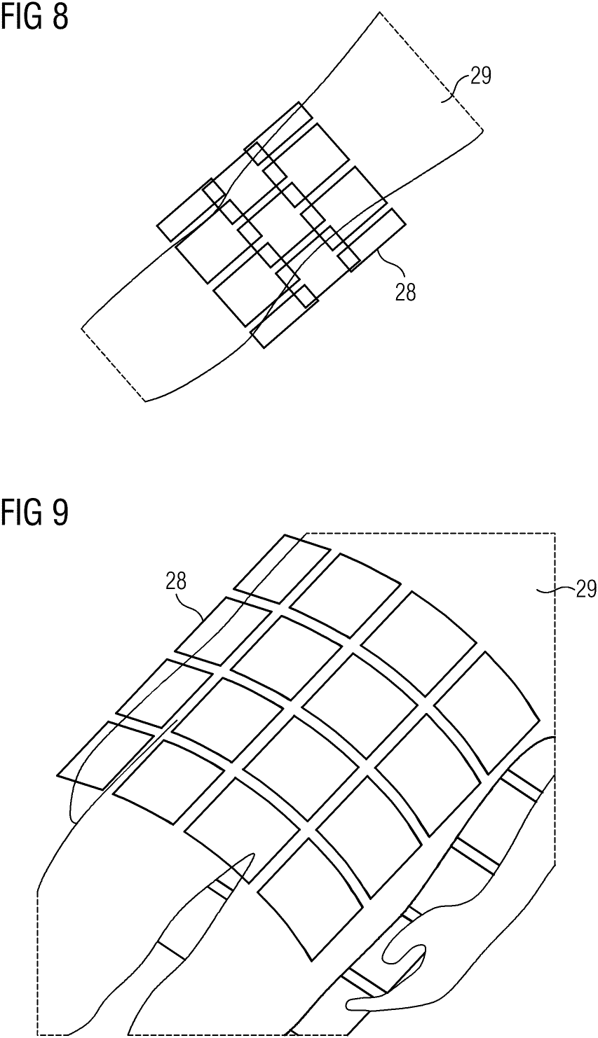

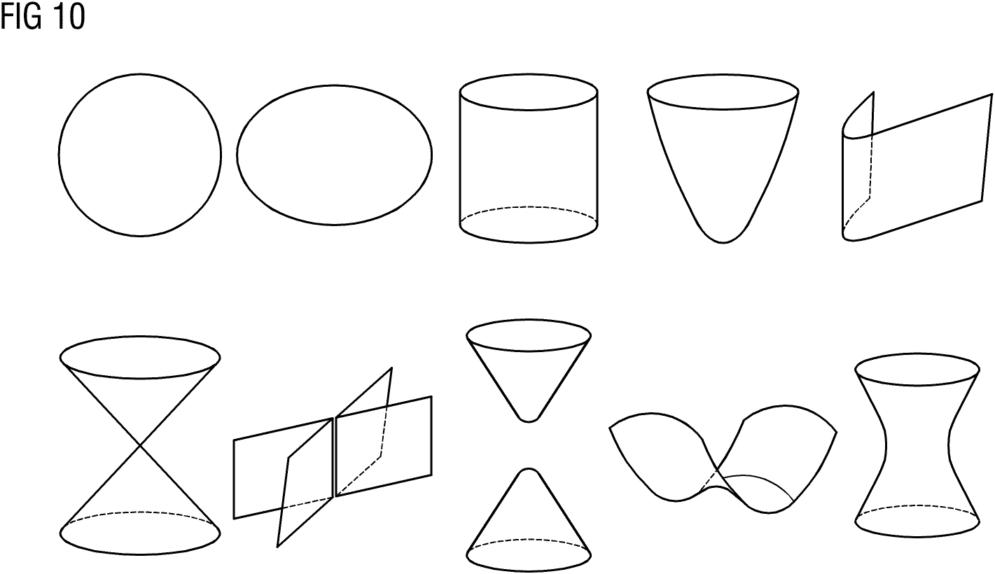

Shown in FIG. 6 is a flexible MR receive coil 28 with a number of receive elements that have corresponding conductor loops 4, where the receive elements may, for example, be embedded in a flexible plastic material 26 and may be connected to a controller 27. FIG. 7 shows schematically a flexible MR receive coil 28 for imaging the head of the patient 29, FIG. 8 shows a flexible MR receive coil 28 for imaging the knee of the patient 29, and FIG. 9 shows a flexible MR receive coil 28 for imaging the abdomen of the patient 29.

In order to describe a flexible MR receive coil 28 mathematically with high degree of accuracy, mathematical models for quadric surfaces may be used. Quadric surfaces include spheres, ellipsoids, cylinders (e.g., circular cylinders or elliptic cylinders), elliptic paraboloids, parabolic cylinders, cones, hyperbolic cylinders, double-layer hyperboloids, hyperbolic paraboloids, single-layer hyperboloids, hyperboloids of one or two sheets, and so forth, as shown schematically in FIG. 10 in order from top left to bottom right. Viewed mathematically a quadric surface is the graph of a second-order equation in the three variables x, y, z. The general form of the equation is:

$$\begin{matrix}\text{A} * {\text{x}}^{2} + \text{B} * {\text{y}}^{2} + \text{C} * {\text{z}}^{2} + \text{D} * \text{x} * \text{y} + \text{E} * \text{y} * \text{z} + \text{F} * \text{x} * \text{z} \\ + \text{G} * \text{x} + \text{H} * \text{y} + \text{I} * \text{z} + \text{J} = 0 ,\end{matrix}$$

where A to J 10 represent coefficients that may be varied to adapt the shape of the coil. Based on this observation, shape and location of the flexible MR receive coil 28 may be determined with the aid of the measured voltages that are induced by temporally variable gradient fields in the receive elements. For example, the following acts may be carried out: a) initialization of the quadric surface to an initial estimated shape by allocation of initial values to the coefficients A to J; b) initialization of the offset (x₀, y₀) of the coil and of the angle of rotation of the coil with regard to the x axis; c) adaptation of the arrangement of the receive elements (e.g., of the conductor loops 4 to the quadric surface); d) computation of the voltages induced in the conductor loops 4, taking into account the current shape of the coil and the gradient strengths as described above; e) use of a gradient descent method in order to adapt the values of the coefficients A to J, the offset (x₀, y₀), and the angle of rotation, so that the mean quadric error between the voltages computed in act d) and the measured voltages is reduced; f) iterative repetition of the acts c), d), and e) until the mean quadric error falls below a specific threshold value.

Different acts of this method may also be further optimized. With a flexible MR receive coil 28 such as is shown in FIG. 6, the initial shape may, for example, be restricted so that the shape corresponds to the surface of a cylinder or of a parabolic cylinder oriented along the axis. A parabolic cylinder that is symmetrical along the x axis is described mathematically by a very simple equation: A*y² = 0.

For other MR receive coils 28, the shape of one or more hyperbolic paraboloids may be more suitable. For this, the simplified equation: A*x² - B*y² + z = 0 applies.

This type of pre-optimization speeds up the speed of conversion of the iterative algorithm, in that the pre-optimization reduces the number of coefficients A to J and sets a starting point that lies closer to the eventual solution. The same consideration applies for the coil offset and the coil rotation. The numerical range in which these parameters may change may be restricted here and, in this way, forces the iterative algorithm to remain close to the eventual solution.

The present embodiments may also be applied for wireless coils that combine an analog-to-digital converter on the coil with a wireless digital transmission.

The methods described above are variable with already known methods for recognition of the patient movement, such as by Hall sensors, 2D or 3D video cameras, or MR movement navigators being able to be combined in order to further refine and to improve the results.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for determining a location of an apparatus inside an imaging volume of a magnetic resonance tomography (MRT) system, wherein the imaging volume is surrounded by a field magnet for creating a static basic magnetic field along a longitudinal axis and by a gradient coil of the MRT system, and wherein the apparatus comprises at least one first conductor loop that runs within a first loop plane, the method comprising:
creating a magnetic alternating field in the imaging volume using the gradient coil;
determining at least one first measured value that depends on a first induction voltage using the at least one first conductor loop, the first induction voltage being induced by a first component of the magnetic alternating field at right angles to the longitudinal axis in the at least one first conductor loop;
determining a location of the apparatus inside the imaging volume at least partly as a function of at least one first measured value and a predetermined magnetic field model for the gradient coil;
detecting a magnetic resonance (MR) signal from an object to be examined in the imaging volume by the at least one first conductor loop; and
creating an MR image as a function of the MR signal.

2. The method of claim 1 wherein to determine the at least one first measured value, the MR signal is suppressed.

3. The method of claim 1, wherein the apparatus is positioned in the imaging volume such that the first loop plane is at least approximately parallel to the longitudinal axis.

4. The method of claim 1, wherein the apparatus further comprises at least one second conductor loop that runs within a second loop plane, and
wherein the method further comprises:
determining, using the at least one second conductor loop, at least one second measured value that depends on a second induction voltage that is induced by a second component of the alternating field at right angles to the longitudinal axis in the at least one second conductor loop; and
determining the location of the apparatus at least partly as a function of the at least one first measured value, the at least one second measured value, and the magnetic field model for the gradient coil.

5. The method of claim 4, wherein the apparatus is positioned in the imaging volume such that the second loop plane is at least approximately parallel to the longitudinal axis.

6. The method of claim 4, wherein the apparatus has at least one third conductor loop that runs within a third loop plane,
wherein the method further comprises:
determining, using the at least one third conductor loop, at least one third measured value that depends on a third induction voltage that is induced by a third component of the alternating field at right angles to the longitudinal axis in the at least one third conductor loop;
determining a first location of the at least one first conductor loop inside the imaging volume at least partly as a function of the at least one first measured value and the magnetic field model;
determining a third location of the at least one third conductor loop inside the imaging volume at least partly as a function of the at least one third measured value and the magnetic field model; and
determining a relative location of the at least one third conductor loop with regard to the at least one first conductor loop as a function of the first location and the third location.

7. The method of claim 2, wherein the apparatus is positioned in the imaging volume such that the first loop plane is at least approximately parallel to the longitudinal axis.

8. The method of claim 7, wherein the apparatus further comprises at least one second conductor loop that runs within a second loop plane, and
wherein the method further comprises:
determining, using the at least one second conductor loop, at least one second measured value that depends on a second induction voltage that is induced by a second component of the alternating field at right angles to the longitudinal axis in the at least one second conductor loop; and
determining the location of the apparatus at least partly as a function of the at least one first measured value, the at least one second measured value, and the magnetic field model for the gradient coil.

9. The method of claim 8, wherein the apparatus is positioned in the imaging volume such that the second loop plane is at least approximately parallel to the longitudinal axis.

10. The method of claim 8, wherein the apparatus has at least one third conductor loop that runs within a third loop plane,
wherein the method further comprises:
determining, using the at least one third conductor loop, at least one third measured value that depends on a third induction voltage that is induced by a third component of the alternating field at right angles to the longitudinal axis in the at least one third conductor loop;
determining a first location of the at least one first conductor loop inside the imaging volume at least partly as a function of the at least one first measured value and the magnetic field model;
determining a third location of the at least one third conductor loop inside the imaging volume at least partly as a function of the at least one third measured value and the magnetic field model; and
determining a relative location of the at least one third conductor loop with regard to the at least one first conductor loop as a function of the first location and the third location.

11. A magnetic resonance tomography (MRT) system comprising:
a field magnet operable to create a static basic magnetic field along a longitudinal axis, and a gradient coil, wherein the field magnet and the gradient coil surround an imaging volume of the MRT system;
an apparatus with at least one first conductor loop that runs within a first loop plane;
a control unit that is configured to activate the gradient coil, such that a magnetic alternating field is created in the imaging volume;
a measurement unit that is connected to the at least one first conductor loop and is configured, as a function of a first induction voltage that is induced by a component of the alternating field at right angles to the longitudinal axis in the at least one first conductor loop, to determine at least one first measured value; and
at least one evaluation unit that is configured to determine a location of the apparatus inside an imaging volume at least partly as a function of at least one first measured value and a predetermined magnetic field model for the gradient coil,
wherein the at least one evaluation unit is configured, depending on a magnetic resonance (MR) signal from an object to be examined in the imaging volume, to create an MR image.

12. The MRT system of claim 11, further comprising a local MR receive coil arrangement that contains the apparatus.

13. The MRT system of claim 12, wherein the local MR receive coil arrangement is configured as a flexible surface coil array.

14. The MRT system of claim 11, further comprising a device for medical treatment of a patient,
wherein the at least one first conductor loop and the device have a predetermined spatial location in relation to one another.

15. The MRT system of claim 11, wherein the apparatus comprises:
a tuning capacitance that is arranged between a first terminal of the at least one first conductor loop and a second terminal of the at least one first conductor loop; and
an inductive component that is arranged electrically in parallel to the tuning capacitance.

16. The MRT system of claim 13, wherein the apparatus comprises:
a tuning capacitance that is arranged between a first terminal of the at least one first conductor loop and a second terminal of the at least one first conductor loop; and
an inductive component that is arranged electrically in parallel to the tuning capacitance.

17. The MRT system of claim 14, wherein the apparatus comprises:
a tuning capacitance that is arranged between a first terminal of the at least one first conductor loop and a second terminal of the at least one first conductor loop; and
an inductive component that is arranged electrically in parallel to the tuning capacitance.

18. The MRT system of claim 15, wherein the measurement unit comprises an amplifier that is connected to the first terminal and the second terminal, and
wherein the measurement unit is configured to provide the at least one measured value at an output of the amplifier, which is connected to the at least one evaluation unit.

19. The MRT system of claim 18, wherein the measurement unit comprises a filter circuit that is arranged between the first terminal and a first input of the amplifier, and between the second terminal and a second input of the amplifier, and
wherein the filter circuit is configured to suppress an MR signal acquired by the at least one first conductor loop.
